(19) United States
(12) Patent Application Publication
(10) Pub. No.: US 2021/0319893 A1
(43) Pub. Date: Oct. 14, 2021

(54) AVATAR ASSISTED TELEMEDICINE PLATFORM SYSTEMS, METHODS FOR PROVIDING SAID SYSTEMS, AND METHODS FOR PROVIDING TELEMEDICINE SERVICES OVER SAID SYSTEMS

(71) Applicant: Vincent S. DeORCHIS, Manhasset, NY (US)

(72) Inventor: Vincent S. DeORCHIS, Manhasset, NY (US)

(21) Appl. No.: 17/106,406

(22) Filed: Nov. 30, 2020

Related U.S. Application Data
(60) Provisional application No. 63/007,420, filed on Apr. 9, 2020.

(51) Int. Cl. G16H 40/67; G16H 80/00; G16H 10/60; G16H 50/20; G16H 50/70; G16H 20/10; G16H 50/50; G16H 40/20; G06N 20/00; A61B 5/00

(52) U.S. Cl. CPC G16H 40/67; G16H 80/00; G16H 10/60; G16H 50/20; G16H 50/70; G16H 20/10; A61B 5/7435; G16H 40/20; G06N 20/00; A61B 5/744; A61B 5/7465; A61B 5/0022; A61B 5/7475; G16H 50/50

(57) ABSTRACT

A avatar assisted telemedicine systems and methods assist during telemedicine services comprising patient examinations. The systems and methods receive input data indicative of a native language of a patient from a patient digital device, render or display at least one communication of a patient digital avatar via a GUI of the patient digital device, wherein the at least one communication is provided in the native language of the patient. Further, the systems and methods assist a queuing of the patient examination over the patient digital device via the at least one communication rendered or displayed by the patient digital avatar. Still further, the systems and methods render, via the patient digital avatar, at least one communication in the native spoken-language of the patient such that the at least one communication assists the patient during at least one portion of the patient examination.

### CROSS-REFERENCE TO RELATED APPLICATIONS

This non-provisional application claims priority to U.S. Provisional Patent Application No. 63/007,420 filed on Apr. 9, 2020, which is incorporated herein by reference.

### SUMMARY OF THE DISCLOSURE

The present disclosure relates to the use of avatar assisted telemedicine platform systems and methods during patient physical examinations. The physical examinations may be, for example, neurologic examinations, National Institute of Health Stroke Scale (hereinafter “NIHSS”), instructions regarding obtaining blood pressures via cuffs, pulse oximetry readings, gait assessments, ophthalmologic examinations, dermatologic assessments, dental examinations, and/or the like. The present disclosure also relates to methods for providing, preparing, and/or utilizing the avatar assisted telemedicine platform systems for providing telemedicine services to patients during patient physical examinations over the avatar assisted telemedicine platform systems.

Described herein are systems and/or methods (hereinafter collectively referred to as “the present systems/methods”) that solve the interaction and/or communication problems between patients and healthcare providers by enabling at least one of a healthcare provider, a patient, and at least one machine learning (hereinafter “ML”) and/or artificial intelligence (hereinafter “AI”) application, software program, and/or tools of the present systems/methods to, remotely or in the presence of the patient, determine, assess, and/or measure one or more physical conditions and/or symptoms of the patient. As a result of the interaction and/or communication problems solved by the present systems/methods, the healthcare provider may medically evaluate and/or diagnosis the patient based on the determined, assessed, and/or measured one or more physical conditions and/or symptoms of the patient.

### BRIEF DESCRIPTION OF THE DRAWINGS

The present disclosure is best understood from the following detailed description when read with the accompanying Figures. It is emphasized that, in accordance with the standard practice in the industry, various features are not drawn to scale. In fact, the dimensions of the various features may be arbitrarily increased or reduced for clarity of discussion.

FIG. 1 is an illustration of a patient interface/display and/or graphic user interface (hereinafter “GUI”) of an avatar assisted telemedicine platform system, according to one or more examples of the disclosure.

FIG. 2 is an illustration of a healthcare provider interface/display and/or GUI of an avatar assisted telemedicine platform system, according to one or more examples of the disclosure.

FIG. 3 is a box diagram illustrating an avatar assisted telemedicine platform system and system components for implementing avatar assisted telemedicine platform methods, according to one or more examples of the disclosure.

### DETAILED DESCRIPTION

As a preliminary matter, it will readily be understood by one having ordinary skill in the relevant art that the present disclosure has broad utility and application. As should be understood, any embodiment may incorporate only one or a plurality of the disclosed aspects herein of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Illustrative examples of the subject matter claimed below will now be disclosed. In the interest of clarity, not all features of an actual implementation are described in this specification. It will be appreciated that in the development of any such actual implementation, numerous implementation-specific decisions may be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

Further, as used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

The present disclosure is directed to digital avatar assisted telemedicine platform and/or interface systems and methods (i.e., the present systems/methods) to facilitate audio and/or visual communication and interaction between one or more users or patients (hereinafter “the patient”) and one or more healthcare providers or examiners (hereinafter “the provider”) during the physical examinations.

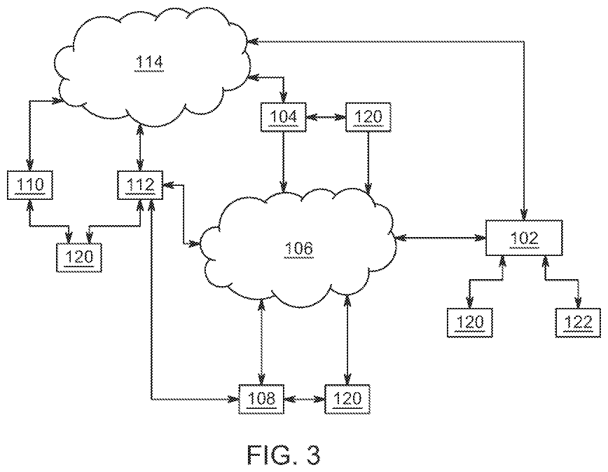

FIG. 3 illustrates a digital avatar assisted telemedicine platform and/or interface system 100 (hereinafter “the system 100”) of the present disclosure. Components of the system 100 may be utilized, accessed, and/or activated to implement one or more digital avatar assigned telemedicine platform and/or interface methods. The system 100 provides, utilizes, and/or facilitates audio and visual interactions and/or communications (hereinafter “the communications”) between the patient and the provider during one or more telemedicine visits and/or the physical examinations. One or more telemedicine platforms and/or interfaces are provided by the system 100 and/or methods such that real-time audio and/or visual teleconferencing between the patient and the provider is facilitated, provided, and/or streamed by the system 100 and/or methods. At least one digital avatar powered and/or provided by the system 100 and/or methods augments the telemedicine platforms and/or interfaces between the patient and the provider. As a result, the real-time audio and/or visual teleconferencing may be augmented by the at least one digital avatar powered and/or provided by the system 100 and/or methods. Augmentation of the communications by the at least one digital avatar may be powered by one or more ML and/or AI applications, software, and/or tools of the system 100. Queuing of the one or more physical examinations may be assisted by the at least one digital avatar powered and/or provided by the system 100. In some embodiments, the queuing of the one or more physical examinations may comprise at least one sequence of at least one of one or more messages and one or more jobs associated with and/or indicative of one or more portions of the one or more physical examinations. held in temporary storage awaiting transmission or processing. Further, the at least one sequence of the one or more messages and/or the one or more jobs may be held in a temporary storage of the system 100 and/or may be awaiting transmission to the patient device 102 and/or processing by the avatar 4 and/or the patient device 102. Subsequent messages and/or subsequent jobs of the sequence may be transmitted to the patient device 102 after prior messages and/or prior jobs are completed or accomplished by each patient during each physical examination. Moreover, the at least one digital avatar may solve the problems associated with the communications between the patient and the provider, such as, for example, communication difficulties, communication differences, written-language differences, spoken-language differences, and/or educational differences between the patient and the provider.

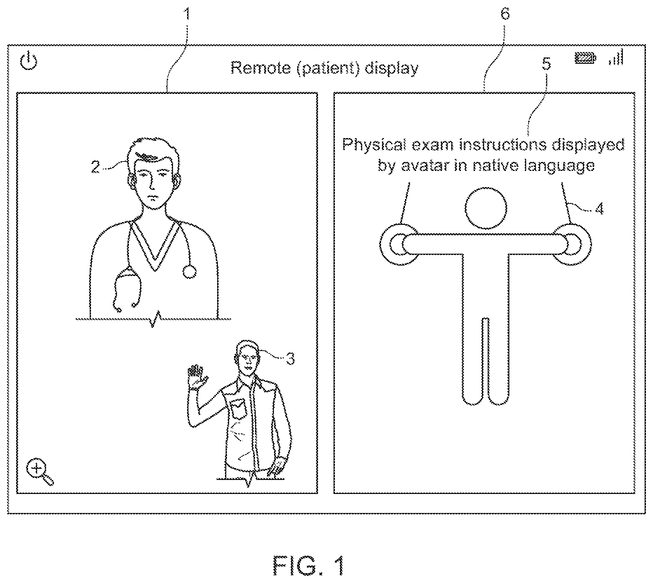

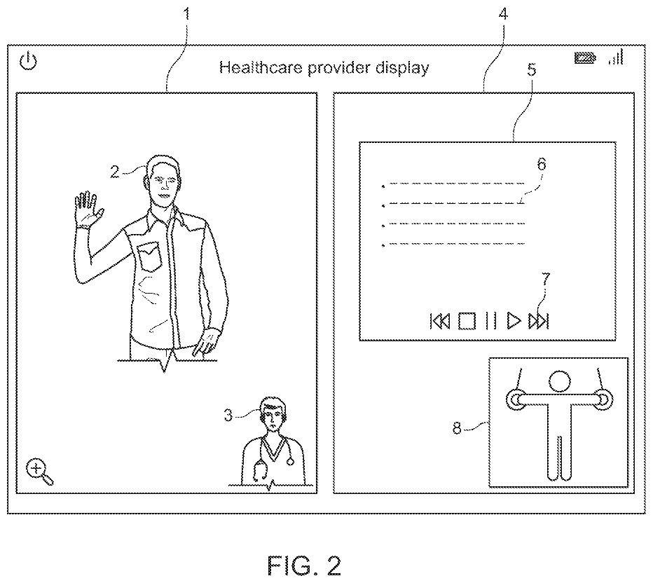

The system 100 and/or methods may comprise at least one first digital device 102 accessible and/or usable by the patient (hereinafter “the patient device 102”) and at least one second digital device 104 accessible and/or usable by the provider (hereinafter “the provider device 104”). The system 100 is configured and/or adapted to facilitate, provide, transmit, and/or receive the communications between the patient device 102 and the provider device 104. Moreover, at least one computer-implemented and/or digital avatar 4 (hereinafter “the avatar 4”), as shown in FIGS. 1 and 2, may be rendered, displayed, implemented and/or accessible via the patient device 102 and/or the provider device 104 (collectively referred to hereinafter as “the devices 102, 104”). The system 100 may provide and/or activate the avatar 4 to solve or address the problems associated with the communication between the patient and the provide via the devices 102, 104.

In embodiments, the devices 102, 104 may be one or more portable digital devices, one or more handheld digital devices, one or more computer terminals or any combination thereof. In embodiments, the devices 102, 104 may be a wired terminal, a wireless terminal or any combination thereof. For example, the devices 102, 104 may be wireless electronic media device, such as, for example, a tablet personal computer (hereinafter “PC”), an ultra-mobile PC, a mobile-based pocket PC, an electronic book computer, a laptop computer, a video game console, a digital projector, a digital television, a digital radio, a media player, a portable media device, a personal digital assistant, an enterprise digital assistant and/or the like. In other embodiments, the devices 102, 104 may be, for example, a hyper local digital device, a location-based digital device, a GPS-based digital device, a mobile device (i.e., a 5G+ mobile device, a 5G mobile device, a 4G mobile device, a 3G mobile device), an ALL-IP electronic device, an information appliance or a personal communicator. The present disclosure should not be deemed as limited to specific embodiments of the devices 102, 104.

The devices 102, 104 may each have at least one display for displaying or rendering information and/or multimedia data stored in a memory or at least one digital storage device accessible by microprocessors (not shown in the drawings) of the devices 102, 104, the communications and/or digital data streamed to the devices 102, 104 via a first digital communication network 106 (hereinafter “the first network 106”), the avatar 4, or a combination thereof. The devices 102, 104 may be in digital communication with each other via or over the first network 106. In an embodiment, one or more digital displays of each of the devices 102, 104 may be or comprise at least one digitized touch-screen and at least one touch-screen graphic user interface (collectively referred to hereinafter as “the GUI”) connected to the microprocessors of the devices 102, 104. In embodiments, the GUI of the patient device 102 may facilitate, permit, and/or allow patient interaction and/or communication by the patient with the patient device 102, and the GUI of the provider device 104 may facilitate, permit, and/or allow provider interaction and/or communication by the provider with the provider device 104.

The GUIs of the devices 102, 104 may facilitate, permit, and/or allow interactions and/or communications with devices 102, 104 by way of or via one or more graphical elements, one or more audio elements, and/or text-based elements. In some embodiments, one or more display links of the one or more audio elements may facilitate, permit, and/or allow interactions and/or communication with the devices 102, 104 via the GUIs of the devices 102, 104. In other embodiments, the GUIs of the devices 102, 104 may facilitate, permit, and/or allow interactions and/or communications with devices 102, 104 by way of or via one or more graphical elements and/or one or more display links, instead of through use of a pure text-based elements or interface. The one or more graphical elements, the one or more text-based elements, and/or the one or more display links may be, may comprise, and/or may include one or more windows, one or more icons, one or more widgets, one or more sliders, one or more text boxes, one or more buttons, one or more menus, one or more screens one or more digital avatars (i.e., the avatar 4), or at least one combination thereof. The one or more graphical elements, the one or more text-based elements, and/or the one or more display links may be selected, highlighted, moved, activated, and/executed through use of the GUIs of the devices 102, 104 and/or via at least one pointing device (i.e., a mouse, a stylus, a digital writing device, a human finger or thumb, or a combination thereof) associated with and/or in digital communication with the devices 102, 104. The displays, the GUIs, and/or the pointing devices of the devises 102, 104 may be configured and adapted to support touch and multi-touch manipulation by the patient and/or the provider. In some embodiments, two or more screens of the GUIs of the devices 102, 104 may be linked together into a workflow of the avatar assisted telemedicine platform systems and methods during patient physical examinations. The workflow and/or navigation between two or more screens of the GUIs of the devices 102, 104 may be facilitated, executed, and/or performed in one or more particular orderings indicative of the patient physical examinations.

The one or more digital displays and/or the GUIs of at least one of the devices 102, 104 may display, render, provide, and/or facilitate the avatar 4, the communications, telemedicine visits, physical examinations, and/or the real-time audio and/or visual teleconferencing between the patient and the provider. Moreover, selectable and/or streaming avatar 4, information, data and/or multimedia data may be rendered, accessed, and/or activated by the devices 102, 104 which may include one or more web sites, one or more web applications, one or more web pages, digital media, one or more IP addresses, audio files or signals, video files or signals, image files or signals, one or more e-mail servers and/or the like.

In embodiments, the devices 102, 104 may have one or more communication components for connecting to and/or communicating with the first network 106. In an embodiment, the one or more communication components of the devices 102, 104 may be a wireless transducer (not shown in the drawings), such as, for example, a wireless sensor network device, such as, for example, a Wi-Fi network device, a wireless ZigBee device, an EnOcean device, an ultra-wideband device, a wireless Bluetooth device, a wireless Local Area Network (hereinafter LAN) accessing device, a wireless IrDA device and/or the like. The present disclosure should not be deemed as limited to specific embodiments of the wireless transducer.

The devices 102, 104 may connect to and/or may access the first network 106 via the one or more communication components of the devices 102, 104. In an embodiment, the devices 102, 104 may be connected to and/or in digital communication with each other via or over the first network 106. In another embodiment, the device 102, 104 may be directly connected to and/or in direct digital communication with each other. In yet another embodiment, a resolver (not shown in the drawings) may be integrated into, or part of, the devices 102, 104. In embodiments, the resolver may be an internet and/or intermediary resolver specifically assigned to the devices 102, 104 and/or provided by an internet service provider of, or associated with, the devices 102, 104.

The devices 102, 104, the resolver, and/or at least one computer server 108 (hereinafter “the server 108”) may be connected to, in digital communication, and/or accessible via the first network 106 of the system 100. As a result, the devices 102, 104 and/or the resolver may be in digital communication with the server 108 and may access at least one internet-accessible resource (hereinafter “internet-accessible resource”) via the first network 106, wherein the internet-accessible resource comprises at least the avatar 4, at least one web site, at least one web page, at least one web application, at least one mobile application, at least one e-mail server, digital information, digital data, digital media content and/or any combination thereof. In embodiments, at least one AI- or ML-based resource may be accessible and/or activatable by the devices 102, 104 and/or the server 108 via the first network 106. In some embodiments, the devices 102, 104 and/or the server 108 may utilize, execute, and/or access the at least AI- or ML-based resource locally or remotely over a cloud server or other digital communication network.

In embodiments, the devices 102, 104, and/or the server 108 may be directly connected and/or in direct digital communication with a database 110 and/or an interface 112. In other embodiments, the devices 102, 104 and/or the server 108 may be connected to the database 110 and/or the interface 112 via a second digital communication network 114 (hereinafter “the second network 114”). The database 110 may be a memory or storage medium that is local with respect to the devices 102, 104, and/or the server 108 or may located remotely with respect to the devices 102, 104 and/or the server 108 whereby “remotely” means positioned at a different physical location than the physical location of the devices 102, 104 and/or the server 108. Similar to the database 110, the interface 112 may be located locally or remotely with respect to the devices 102, 104 and/or the server 108. In an embodiment, the system 100 and/or the database 110 may comprise one or more additional systems and/or may be distributed across multiple servers and/or datacenters (not shown in the drawings). The at least one AI- and/or ML-based resource may be accessible and/or activatable by the devices 102, 104 and/or the server 108 via the database 110 and/or the interface 112 over the first network 106 and/or the second network 114. The devices 102, 104, the server 108, the database 110, and/or the interface 112 may be digitally connected and/or in digital communication with each other via the first network 106 and/or the second network 114.

A memory, digital storage device and/or non-transitory computer-readable medium, which may be accessed and/or executed by a microprocessor incorporated into or included within the system 100, the devices 102, 104, the server 108 and/or the interface 112, may have stored thereon executable computer-implemented instructions, computer programs, one or more algorithms and/or software that, when executed by the microprocessor, perform one or more computer-implemented steps of the present methods disclosed herein. In embodiments, the executable instructions, computer programs, algorithms, and/or software may be AI- and/or ML-based or optimized executable instructions, computer programs, algorithms, and/or software. The present AI applications, software, and/or tools may be executed by the devices 102, 104, the server 108 and/or the interface 112 via the AI- and/or ML-based or optimized executable instructions, computer programs, algorithms, and/or software. In some embodiments, the AI- and/or ML based or optimized executable instructions, computer programs, algorithms, and/or software may be accessible and/or executable locally with respect to the devices 102, 104, the server 108, and/or the interface 112. In some embodiments, the GUIs of the devices 102, 104 may be web-based, for example, with one or more parts of one or more pages being loaded from the 108 server, the database 110, and/or the interface 112, or may natively-compiled to execute on at least one of the devices 102, 104, even when the first network 106 and/or the second network 114 (collectively referred to hereinafter as “networks 106, 114”) may not be or are not available to the devices 102, 104.

In embodiments, the networks 106, 114 may be, for example, a personal area network (PAN), a local area network (LAN), a campus area network (CAN), a Metropolitan area network (MAN), a wide area network (WAN) and/or the like. In an embodiment, the networks 106, 114 may be a wireless network, such as, for example, a 5G+ network, a 5G network, a 4G network, a 3G network, a wireless MAN, a wireless LAN, a wireless PAN, a Wi-Fi network, a WiMAX network, a global standard network, a personal communication system network, a pager-based service network, a general packet radio service, a universal mobile telephone service network, a radio access network and/or the like. In an embodiment, the networks 106, 114 may be a fixed network, such as, for example, an optical fiber network, an Ethernet, a cabled network, a permanent network, a power line communication network and/or the like. In another embodiment, the networks 106, 114 may be a temporary network, such as, for example, a modem network, a null modem network and/or the like. In yet another embodiment, the networks 106, 114 may be an intranet, extranet or the Internet which may also include the world wide web. The present disclosure should not be limited to a specific embodiment of the networks 106, 114.

The present disclosure should not be deemed as limited to a specific number of digital devices, computer servers, databases, digital communication networks, resolvers and user interfaces which may access and/or may utilize the present systems/methods disclosed herein. The present systems/methods disclosed herein may include and/or incorporate any number of digital devices, computer servers, databases, digital communication networks, resolvers and/or user interfaces as known to one of ordinary skill in the art.

The at least one AI- and/or ML-based resource of the system 100 includes one or more techniques that enable one or more machines or computers of the system 100 (i.e., the devices 102, 104, the server 108, the database 110, and/or the interface 112) to mimic human behavior. In embodiments, the one more techniques may comprise machine ML techniques which are a subset of AI comprising one or more statistical methods to enable the one or more machines or computers of the system 100 to improve with experience. The one or more statistical methods of the present systems/methods are at least one method selected from supervised learning, unsupervised learning, semi-supervised learning, reinforcement learning, deep learning, and at least one combination thereof. Supervised learning may comprise at least one selected from regression, classification, and at least one combination thereof. Unsupervised learning may comprise at least one selected from clustering, dimensionality reduction, and at least one combination thereof. Semi-supervised learning may comprise at least one selected from self-training, one or more low density separation models, one or more graph-based algorithms, and at least one combination thereof. Reinforcement learning may comprise at least one selected from dynamic programming, one or more Monte Carlo methods, one or more Heuristic methods, and at least one combination thereof. Deep learning is a subset of the ML that is configured, adapted, or programmed to make computations of one or more multi-layer neural networks feasible. Deep learning may comprise at least one artificial neural network selected from at least one recurrent neural network (hereinafter “the RNN”), at least one convolutional neural network, and at least one combination thereof.

Architectures of the at least one AI- and/or ML-based resource disclosed herein comprises at least one of the RNN and AI software 120 (hereinafter “the AI software 120”) as shown in FIG. 3. In some embodiments, the at least one AI- and/or ML-based resource and/or the AI software 120 may be web-based, for example, with one or more parts of AI software 120 being loaded from and/or executable via the 108 server, the database 110, and/or the interface 112. Further, the at least one AI- and/or ML-based resource and/or the AI software 120 may locally- or natively-compiled to execute on at least one of the devices 102, 104, even when the networks 106, 114 may not be or are not available to the devices 102, 104. Alternatively, the at least one AI- and/or ML-based resource and/or the AI software 120 may be a combination of web-based and locally- or natively-compiled such that the devices 102, 104 and the AI software 120 require fewer computing assets or resources and/or improve AI processing speeds achievable by the system 100 and/or methods disclosed herein.

The AI software 120 of the present systems/methods disclosed herein may comprise and/or include at least one classifier, model, or network selected from a fully recurrent network, at least one Elman network, at least one Jordan network, a Bayesian network, a Hopfield network, an Echo state network, an independently RNN, a recursive network, a neural history compressor network, a second order RNN, a long short-term memory (hereinafter “LSTM”) network, a gated recurrent unit network, a bi-directional LSTM network, a continuous-time network, a hierarchical network, a recurrent multilayer perceptron network, a multiple timescales model network, at least one neural Turing machine, a differentiable neural computer network, a neural network pushdown automata, at least one memristive network, and at least one combination thereof. In embodiments, the at least one classifier, model, or network may be at least one neural network with a non-conventional number of nodes at one or more layers and/or may comprise one or more hidden layers.

The AI software 120 of the present systems/methods may be accessed, utilized, activated, and/or implemented by at least one of the devices 102, 104, the server 108, the database 110, the interface 112, and one or more combinations thereof. As a result, the AI software 120 of the present systems/methods may solve the problems associated with the communications between the patient and the provider via GUIs of the devices 102,104 over the networks 106, 114. Further, the AI software 120 of the present systems/methods may power and/or facilitate the avatar 4 on the GUIs such that the avatar 4 may solve the problems associated with the communications between the patient and the provider via the GUIs of the devices 102, 104.

The present AI applications activatable by the AI software 120 of the present systems/methods may comprise and/or include at least one selected from a machine translation, a robot control, a time series prediction, a speech recognition, a speech synthesis, a time series anomaly detection, a rhythm learning, a music composition, a grammar learning, a handwriting recognition, a human action recognition, predicting or forecasting one or more medical events, and at least one combination thereof. The LSTM network(s) of the system 100 and/or methods disclosed herein are suited for and/or capable of classifying, processing and making predictions based on time-series data, since there can be lags of unknown duration between important events in a time series.

A time series of the system 100 and methods disclosed herein is a series of data points indexed, listed, and/or graphed in time order. Additionally, a time series is a sequence taken at successive equally spaced points in time. In embodiments, the present time series may be a sequence taken successively unequal spaced points of time. The spaced points of time may be a plurality of time points during the communications between the patient and the provider via the GUIs of the devices 102, 104. The present AI analysis of the time-series data provided by the AI software 120 comprises methods for analyzing time-series data to extract or determine meaningful statistics and/or other characteristics of or associated with the time-series data. Time series forecasting of the present systems/methods utilizes a model to predict future values based on observed values of or associated with the time-series data gathered, collected, and/or observed over the system 100 and/or during the methods disclosed herein. Further, the time-series data may be gathered, collected, and/or observed by the present systems/methods during the communications between the patient and the provider via the GUIs of the devices 102, 104 over the networks 106, 114.

The present AI applications and/or tools of the AI software 120 for investigating and/or analyzing time-series data may include AI and/or ML models or techniques utilizing one or more artificial neural networks. For example, at least one RNN has been developed, trained, and/or applied to the time-series data gathered, collected, and/or observed via the patient device 102 during the communications between the devices 102, 104 via the GUIs. In some embodiment, training of the AI applications and/or tools of the AI software 120 may be tagged input-output pairs or unsupervised learning by at least one of the devices 102, 104, the server 108, the interface 112, or at least one combination thereof. The present AI and/or ML models and techniques are trained such that selection of the output results improve both speed or accuracy of the AI applications and/or tools of the AI software 120. The AI and/or ML learning models and techniques of the present systems/methods generate and filter training data results such that present AI applications and/or tools of the AI software 120 and/or the devices 102, 104 require fewer computing assets or resources and/or improve AI processing speeds achievable by the system 100 and/or methods disclosed herein.

In embodiments, applying the present RNN of the AI software 120 to the time-series data may comprise adding or appending time-related information of the time-series data to numerical vectors and/or code embeddings (“code embeddings”). The time-series data may comprise streaming health data for a first point of time and at least one second point of time during the communications via the GUI of at least the patient device 102. The at least one second point of time is subsequent to the first point of time during the communications via the GUIs and may comprise a plurality of second points of time subsequent to the first point of time during the communications via the GUIs. The streaming health data of the time-series data from at least the patient device 102 may comprise at least one of streaming audio data, streaming video data, image data, text-based data, or a combination thereof. The time-series data and/or the streaming health data is input data from the at least the patient device 102 for the present AI applications and/or tools of the AI software 120 and may be obtained or gathered by one or more types of healthcare sensors 122 (hereinafter “the sensors 122”) associated with and/or in digital communication with at least the patient device 102 or the GUI of the patient device 102. In embodiments, the sensors 122 comprise at least one digital camera or video recorder, at least one digital microphone or audio recorder, an imagine device, a measuring device, a diagnostic device, or a combination thereof. The input data from the sensors 122 may represent Boolean values, letters, words, sentences, paragraphs, symbols, gestures, body movements, conversations, images, videos, examinations, demonstrations, measurements, readings, determinations, or a combination thereof. After being obtained or gathered by the sensors 122, the input data may be filtered, handled, parsed, partitioned, pre-processed, and/or separated into more than one component prior to utilization by the present AI applications and/or tools of the AI software 120. The AI software 120 of present systems/methods may utilize the more than one component of the input data to provide, generate, calculate, produce output data for the system 100.

The code embeddings are used by the RNN of the AI software 120 to represent time-stamped codes for the time-series data that cover one or more observed medical conditions (“observations”) exhibited by or associated with the patient. The observations may by indicative of health and/or medical conditions and/or symptoms of the patient utilizing the patient device 102 and the present AI applications and/or tools of the AI software 120 may assign weight values to one or more aspects or features of the conditions and/or symptoms of the patient. The streaming health data of the time-series data from the patient device 102 may cover and/or be indicative of the observations such that the RNN processes the streaming health data to generate the code embeddings representative of the time-stamped codes for the observations. In embodiments, the streaming audio date, the streaming video data, image data, text-based data or the combination thereof from the patient device 102 via the GUI is processed by the RNN to generate the code embeddings covering the observations of the time-series data. Further, a code embedding LSTM neural network may generate the code embeddings based on at least one of the time-series data, the streaming health data from the sensors 122, the patient device 102, the observations, or a combination thereof. Each code embedding may be indicative or representative of each observations processed by the RNN or the code embedding LSTM neural network of the AI software 120. Further, each code embedding may be assigned or associated with a code weight based on each observation of the time-series data.

The RNN and/or the code embedding LSTM neural network of the AI software 120 may have been trained with the code weights for the observations and/or each of the code weights for each of the observations may have trained in the RNN and/or the code embedding LSTM neural network of the AI software 120. In an embodiment, the code weights of the observations, that need to be learned during the training, determine how one or more gates may operate during the generation of the code embeddings. At least one of a deep learning platform, a deep learning framework, and/or a deep learning library of the system 100 may be utilized for training and/or deploying the RNN and/or the code embedding LSTM neural network of the AI software 120 and for supporting one or more AI and/or ML algorithms for generating each of the code embeddings for each of the observations. The deep learning platform, framework, and/or library may define each code weight indicative of each observation of the streaming health data and/or the time-series data received from the patient device 102 via the GUI and/or the sensors 122. In embodiments, the deep learning platform, framework, and/or library of the system 100 may define each native spoken-language associated with and/or indicative of the streaming audio data of the streaming health data and/or the time-series data. Moreover, the deep learning platform, framework, and/or library may define each native written-language associated with and/or indicative of the image data, the text data, and/or text-based data received from the patient device 102 via the GUI and/or the sensors 122.

By utilizing the code embeddings generated by the code embedding LSTM neural network of the AI software 120, the RNN of the AI software 120 may provide one or more results, predictions, and/or forecasts based on the streaming health data received from the patient device 102 via the GUI and/or the sensors 122. In embodiments, the one or more results, predictions, and/or forecasts may include one or more multi-label predictions that are clinically meaningful with respect to the patient. In embodiments, the multi-label predictions may comprise or include a past, present or current, and/or a future status of the patient utilizing the present systems/methods disclosed herein. The multi-label predictions provided by the AI software 120 may also predict and/or forecast one or more future health events of the patient, at least one medical or health diagnosis of the patient, at least one medication order or prescription for the patient, and/or one or more future medical treatments, observations, and/or examinations.

In some embodiments, the one or more results, predictions, and/or forecasts provided by the RNN of the AI software 120 may automate at least one medical detection, monitoring, therapy prediction, and therapy response for the patient. In these embodiments, the present RNN is a prediction RNN configured, adapted and/or trained to process the generated code embeddings to provide, produce, and/or generate a RNN output of the AI software 120. Further, the RNN output of the prediction RNN of the system 100 and/or the AI software 120 may comprise the one or more classifications, likelihoods, results, recommendations, predictions, and/or forecasts of or associated with the patient or the health or medical condition(s) of the patient. Still further, the RNN output of the system 100 and/or the AI software 120 may be based on or generated from at least one weight calculation of the code weights of the code embeddings that cover the observations of the time-series data. In an embodiments, the RNN output of the system 100 and/or the AI software 120 may to control, actuate, and/or manipulate one or more health or medical devices, sensors, applicators, dispensers, and/or healthcare-related machines.

In one embodiment, the weight calculation may be an estimate or forecast based on the code weights of the code embeddings. In another embodiment, a mathematical process of finding, identifying, and/or determining an amount or a number based on the code weights generates the weight calculation and/or the RNN output of the system 100 and/or the AI software 120. In other embodiments, the weight calculation comprises adding, subtracting, multiplying, and/or dividing the code weights to generate the RNN output of the system 100 and/or the AI software 120. Still further, the weight calculation may be at least one arithmetical calculation using at least one algorithm of the present AI applications or tools and/or the AI software 120. For example, weight calculation may be computation comprising both arithmetical and non-arithmetical steps according to specifically defined model, such as, for example, the at least one algorithm of the AI applications or tools and/or the AI software 120. As a result, the present AI applications or tools and/or the AI software 120 achieve improved AI speeds and accuracies by eliminating subjectivity and/or introducing an objective decision making process based on weight calculations and the specifically defined model set forth in the at least one algorithm of the present AI applications or tools and/or the AI software 120.

In embodiments, the present AI applications or tools and/or the AI software 120 may be performed, executed, and/or implemented either locally or remotely with respect to at least one of the devices 102, 104, the server 108, the database 110, the interface 112, and/or the sensors 122. For example, the present AI applications or tools and/or the AI software 120 may be performed, executed, and/or implemented locally or what is known as IA-on-the-edge. Alternatively, the present AI applications, software, and/or tools may be performed, executed, and/or implemented in a cloud server system or at another remote location.

In embodiments, the present AI applications or tools and/or the AI software 120 may be performed, executed, and/or implemented on at least one AI- and/or ML-based hardware (hereinafter “the AI-based hardware”). At least one of the devices 102, 104, the server 108, the database 110, the interface 112, and/or the sensors 122 may comprise, implement, and/or include the AI-based hardware. Further, the devices 102, 104, the server 108, the database 110, the interface 112, and/or the sensors 122 may be in digital communication with the AI-based hardware. In some embodiments, the AI-based hardware may be accessed and/or activated by the devices 102, 104, the server 108, the database 110, the interface 112, and/or the sensors 122 to implement, execute and/or utilize the AI software 120 of the system 100.

The AI-based hardware of the system 100 may comprise or consist of at least one AI- or ML-based central processing unit (hereinafter “CPU”), at least one AI- or ML-based graphics processing unit (hereinafter “GPU”), at least one AI- or ML-based integrated graphics processor (hereinafter “IGP”), or at least one combination thereof. In embodiments, the AI-based hardware may comprise at least one AI-specific and/or AI-optimized CPU, GPU, IGP, or at least one combination thereof. For example, the AI-based hardware of the system 100 may be AI-specific integrated circuits and/or AI-optimized GPUs. Further, the AI-based hardware of the system 100 may comprise one or more analog AI cores, one or more AI-optimized systems, one or more digital AI cores, heterogeneous integration, machine intelligence, ML quantum computing, or at least one combination thereof. Moreover, the AI-based hardware of the system 100 may comprise one or more AI accelerators and/or may be configured and/or adapted such that performance, execution, and/or implementation of the present AI applications tools and/or the AI software 120 may be improved, accelerated and/or increased by the AI-based hardware of the system 100.

The system 100 and/or the methods disclosed herein may provide the one or more telemedicine platforms and/or interfaces that allow for the communications and/or real-time audio and/or visual teleconferencing (hereinafter “real-time teleconference”) between the devices 102, 104 via the GUIs and/or displays of the devices 102, 104, the sensors 122, or a combination thereof over the networks 106, 114. The one or more telemedicine platforms and/or interfaces and/or the communications provided by the system 100 and/or present methods disclosed herein are augmented by the avatar 4 assisted queuing of the one or more physical examinations via the GUIs and/or displays of the devices 102, 104, the sensors 122, or a combination thereof. In embodiments, the avatar 4 may be a 3D digital avatar and/or may be powered or implemented via the AI software 120 and/or the AI-based hardware of the system 100.

In embodiments, the one or more physical examinations assisted by the avatar 4 via the GUIs and/or displays of the devices 102, 104 may comprise at least one of a neurologic examination, a NIHSS, one or more instructions regarding obtaining blood pressures via cuffs, a pulse oximetry reading, a gait assessment, an ophthalmologic examination, a dermatologic assessment, a dental examination, and/or other medical- or health-related examination. The present systems and methods may provide one or more downloadable computer-implemented telemedicine applications (hereinafter “the telemedicine application”) which may be executed, implemented and/or performed on at least one of the devices 102, 104. The assistance provided to the patient by the avatar 4 via the GUI and/or displays of the patent device 102 may be provided in the native spoken-language of the patient, the native written-language of the patient, or a combination thereof. Assistance provided to the provider by the avatar 4 via the GUI and/or displays of the provider device 104 may be provided in the native spoken-language of the provider, the native written-language of the provider, or a combination thereof.

The native spoken- and/or written-language of the patient may be different than the native spoken- and/or written-language of the provider. The AI software 120 of the system 100 may determine, identify, and/or select the native spoken- and/or written-language of the patient based on the streaming health data received from the patient device 102 via the GUI and/or displays of the patient device 102 and/or the sensors 122. The native spoke- and/or written language of the provider may be determined, identified, and/or selected by the AI software 120 based on streaming data or inputted data received from or collected by the provider device 104. In an embodiment, the patient may select the native spoken- and/or written-language of the patient to be utilized by the system 100 by inputting data or information into the GUI and/or displays of the patient device 102. Further, the provider may select the native spoken- and/or written-language of the provider to be utilized by the system 100 by inputting data or information into the GUI and/or displays of the provider device 104. Still further, the AI software 120 may translate and/or interpretate between the native spoken- and/or written-language of the patient and the native spoken- and/or written-language of the provider. Moreover, the AI software 120 may provide language translations and/or language interpretations to the patient and the provider via the avatar 4, the GUIs and/or displays of the devices 102, 104.

In embodiments, the provider utilizes a healthcare provider and/or examiner interface/display (hereinafter “the provider display”) in the form of or taking the form as the GUI or displays of the provider device 104, as shown in FIG. 2, over or on the telemedicine application to control at least one remotely displayed avatar (i.e., the avatar 4) which may depict one or more portions of the one or more physical examinations via the GUI or displays of the patient device 102 for the patient to mimic or execute, and/or may provide patient instructions to use one or more healthcare assisted physiologic monitoring devices (hereinafter “the healthcare device”) over a remote and/or patient interface/display (hereinafter “the patient display”) in the form of or taking the form as the GUI or displays of the patient device 102, as shown in FIG. 1. In some embodiments, the healthcare device comprises, consists of, and/or includes the sensors 122.

In embodiments, the healthcare device and/or the sensors 122 of the system 100 may comprise or include at least one of blood pressure cuff or sphygmomanometer, pulse oximetry, glucometer, EEG/EKG electrode hook up, infrared thermometer, and/or other health- or medical-related monitoring devices.

FIG. 1 illustrates the GUI and/or displays of the patient device 102 in the present systems/methods disclosed herein. The GUI and/or displays of the patient device 102 may display and/or render at least one first image 2 of the provider or a health examiner (collectively referred to hereinafter as “the provider”) and at least one second image 3 of the patient or a user of the system 100 (collectively referred to hereinafter as “the patient”). The first and second images 2, 3 comprise one or more video images of the provider on the patient device 102 and one or more video images of the patient as seen from a front facing camera and/or sensor of the patient device 102 of the patient.

The GUI and/or displays on the patient device 102, as shown in FIG. 1, may also comprise the avatar 4 which may depict, show, and/or visualize at least one portion of one or more physical examinations to be mimicked by the patient. Further, the GUI and/or displays of the patient device 102 may comprise one or more written instructions 5 (hereinafter “the instructions 5”) in a native written-language of the patient. The instructions 5 may disclose, detail, and/or explain the at least one portion of the one or more physical examinations. Additionally, the avatar 4 and/or the instructions 5 may be used or utilized by the provider to evaluate for one or more language impairments of the patient. An avatar user interface window may be provided on the GUI and/or displays of the patient device 102 and/or may contain, display, and/or render the avatar 4 and/or the instructions 5. In embodiments, the avatar 4 and/or the instructions 5 may be powered by AI software 120 and/or the AI-based hardware of the present systems/methods disclosed herein. In an embodiment, the avatar 4 is an interactive 3D digital avatar that may be powered by the AI software 120 of the system 100.

FIG. 2 illustrates the GUI and/or displays of the provider device 104 provided by the present systems/methods disclosed herein. The GUI and/or displays of the provider device 104 may comprise a user interface that displays the first and second images 2, 3. The first and second images 2, 3 displayed or rendered on the GUI and/or displays of the provider device 104 may comprise one or more video images of the patient on the GUI and/or displays of provider device 104 and/or one or more video images of the provider on the GUI and/or displays of provider device 104 as seen from the telehealth front facing camera of the provider device 104. The GUI and/or displays of provider device 104 may also comprise a remote telehealth station control interface window 1 and/or a physical exam control interface sub-window comprising the avatar 4 and/or the instructions 5.

The GUI and/or displays of the provider device 104, as shown in FIG. 2, may further comprise one or more segments 6 of the physical exam to be presented to the patient on remote telehealth station via the GUI and/or displays of patient device shown in FIG. 1. Additionally, the GUI and/or displays of the provider device 104 may comprise a control panel 7 for management of one or more segments 6 of the physical examination and/or the avatar 4 which may include at least one of play, repeat, next and previous exam portions as well as selection of patient's preferred or native spoken- and/or written-language for one or more visual and/or auditory queues. Moreover, the GUI and/or displays of provider device may comprise at least one image 8 of the avatar 4 being displayed to the patient via the GUI and/or displays of the patient device 104.

In embodiments, the present systems/methods disclosed herein provide, comprise and/or utilize the AI software 120 as shown in FIG. 3. The GUI and/or displays of the provider device 104, as shown in FIG. 2, may comprise one or more viewing windows that display, render, show one or more metrics of the patient that was measured, determined, and/or identified by the AI software 120 (hereinafter “AI-measured metrics”). Additionally, AI-measured metrics may provide the provider with timely and accurate evaluation, diagnosis, prediction, forecast and/or health status of the patient via the GUI and/or displays of the provider device 104.

In embodiments, the avatar 4 may be presented to the patient along with written, visual, and/or auditory explanations in the native spoke- and/or written-language of the patient which may be determined, identified, and/or selected by the AI software 120, the streaming health data, and/or inputted data received by the devices 102, 104.

In embodiments, the present systems/methods disclosed herein will be further augmented by the AI software 120 which may quantize and interpret the visual data (i.e., the streaming health data) as well as enhance the quality of said visual data with measurements made via one or more additional sensors (i.e., the sensors 122). In some embodiments, the one or more additional sensors and/or sensors 122 may comprise Light Detection and Ranging or LIDAR, gyroscopes, and accelerometers. The analysis of the visual data or streaming health data by the AI software 120 may be performed on the patient device 102 so as to avoid at least one artifact being introduced by poor network connections via the networks 106, 114.

Extraocular movements of the patient may be analyzed to assess differences in horizontal, vertical and rotatory velocity as well as the presence of dysconjugate gaze via the AI software 120. In an embodiment, the AI software 120 may analyze the extraocular movements of the patient during the communications between the patient and the provider via the devices 102, 104. The present systems/methods disclosed herein may measure a change in pupillary response to light and determine a presence of a relative afferent pupillary defect or RAPD via the AI software 120. In embodiment, the AI software 120 may measure the change and/or determine the presence of RAPD. Visual acuity can be determined by response to interactive remotely displayed eye chart in the native written-language of the patient and/or via the AI software 120.

At least one lid assessment, controlled by the AI software 120, may assess distance from superior pupillary rim to evaluate for the presence of ptosis and lid lag. At least one fundus photography, interpreted by the AI software 120, may be performed to assess for presence of retinal pathology such as arterial/venous occlusion, hemorrhage, macular degeneration, retinopathy, and neoplasm.

Facial movements during neurologic examination will be examined for facial asymmetry and weakness (paresis) of movement. In embodiments, the AI software 120 may analyze the facial movements during the neurologic examinations. Further analysis of discrepancies in lower vs. upper facial muscle movements can provide determination of upper (central nervous system) vs. lower (peripheral nervous system) motor neuron dysfunction/localization. In an embodiment, the AI software 120 may analysis the facial movements and/or the discrepancies.

Measurement of limb stability on elevation may be used to assess for pronation and drift, two findings associated with symptoms of mild stroke. Additionally, as quantification of manual motor testing remotely via telehealth can be challenging, the AI software 120 may assess velocity of drift and pronation as well as height of elevation above the body to estimate degree of limb weakness. Assessments of dysmetria (i.e., finger-nose-finger and heel-to-shin) may be analyzed for tremor/jitter via the AI software 120. In an embodiment, the AI software 120 may analysis the measurement of limb stability and assessments of dysmetria.

Language function may be analyzed by the AI software 120 to assess for presence of dysarthric (slurred) and aphasic/dysphasic (impaired language) speech patterns, characterize the pattern and track improvement or deterioration over time.

Additional physiologic monitoring devices can be connected to the telehealth device to better quantify metrics such as strength, intra-ocular pressure (10P), hearing acuity and balance. In an embodiment, the sensors 122 may comprise, consist of, and/or include the additional physiologic monitoring devices and/or the AI software 120 may quantify the metrics.

The AI software 120 may be utilized for document capture. Patient's with medical records will display those records to the remote camera of the patient device 102. The system 100 and/or the AI software 120 will detect and identify those images as documents and then capture and convert the images or documents to at least one user friendly format such as PDF. The convert images or documents may be presented to the GUI and/or displays of the provider device 104 for immediate review and/or potential or subsequent storage. The images or documents may also be translated into the native written-language of the provider during this process via an optical character recognition (OCR) related process and/or the AI software 120.

One or more metrics related to, determined with, identified with generated by, predicted by, and/or forecasted by the AI software 120 may be exportable in at least one preferred document format of the provider so as to be included in the electronic health record (EHR) of the patient.

The present avatar assisted telemedicine systems and methods may enhance the healthcare provider-patient encounter especially in settings where communication may be obstructed by loud ambient noise such as in an ICU, emergency room or battlefield. Additionally, the present avatar assisted telemedicine systems and methods may overcome one or more barriers in language differences and/or enhance interpretation of visual information, especially when poor network connectivity of the networks 106, 114 may result in degraded image/audio quality communications between the devices 102, 104.

Examples in the present systems/methods disclosed herein may also be directed to a non-transitory computer-readable medium that stores computer-executable instructions and/or the AI software 120, which are executable by one or more processors of at least one of the devices 102, 104, the server 108, and/or the interface 112 from which the computer-readable medium is accessed. A computer-readable media may be any available media that may be accessed by at least one of the devices 102, 104, the server 108, and/or the interface 112. By way of example, such computer-readable media may comprise RAM, ROM, EEPROM, CD-ROM or other optical disk storage, magnetic disk storage or other magnetic storage devices, or any other medium that may be used to carry or store desired program code in the form of computer-executable instructions, the AI software 120, and/or data structures and that may be accessed by at least one of the devices 102, 104, the server 108, and/or the interface 112. Disk and disc, as used herein, includes compact disc, laser disc, optical disc, digital versatile disc, floppy disk and Blu-ray® disc where disks usually reproduce data magnetically, while discs reproduce data optically with lasers.

Note also that the software implemented aspects of the present systems/method disclosed herein are usually encoded on some form of program storage medium or implemented over some type of transmission medium. For example, the software 120 may be encoded on a form of program storage medium or implemented over a type of transmission medium. The program storage medium is a non-transitory medium and may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The claimed subject matter is not limited by these aspects of any given implementation.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Obviously, many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

What is claimed is:

1. An avatar assisted telemedicine platform system configured to provide telemedicine services comprising a patient examination, the system comprising:
a patient GUI displayed or rendered on a patient digital device;
a provider GUI displayed or rendered on a provider digital device;
a digital communication network connected to the patient digital device and the provider digital device such that patient digital device and the provider digital device are in digital communication with each other via the digital communication network,
a patient digital avatar displayed or rendered on the patient GUI;
input data indicative of a native language of a patient and received from the patient digital device or the digital device; and
at least one communication in the native language of the patient and provided or rendered by the patient digital avatar such that the at least one communication of the patient digital avatar assists the patient during at least one portion of the patient examination.

2. The avatar assisted telemedicine platform system of claim 1, wherein the at least one communication in the native language of the patient comprises at least instruction in the native spoken-language of the patient and directed to the at least one portion of the patient examination.

3. The avatar assisted telemedicine platform system of claim 1, wherein the input data indicative of the native language of the patient comprises streaming audio data received from the patient digital device or a selected language inputted into the patient digital device.

4. The avatar assisted telemedicine platform system of claim 3, further comprising:
artificial intelligence (AI) software executable by at least one of the patient digital device and the provider digital device and configured to determine the native language of the patient based on the streaming audio data received from the patient digital device.

5. The avatar assisted telemedicine platform system of claim 4, further comprising:
AI-based hardware executing and/or implementing the AI software to determine, identify, and/or select the native language of the patient based on the streaming audio data, wherein the AI-based hardware consists of at least one selected from an AI-optimized CPU, an AI-optimized GPU, an AI-optimized IGP, an AI-specific accelerator, an AI-specific integrated circuit, and at least one combination thereof.

6. The avatar assisted telemedicine platform system of claim 1, further comprising:
at least one sensor or monitoring device in communication with the patient digital device and configured for streaming healthcare data of the patient collected during the at least one portion of the patient examination.

7. The avatar assisted telemedicine platform system of claim 6, further comprising:
artificial intelligence (AI) software for processing and/or analyzing the streaming healthcare data of the patient collected during the at least one portion of the patient examination.

8. The avatar assisted telemedicine platform system of claim 7, further comprising:
time-series data consisting of a series of data points indexed, listed, and/or graphed in time order by the AI software, wherein the series of data points are based on the streaming healthcare data of the patient collected during the at least one portion of the patient examination.

9. The avatar assisted telemedicine platform system of claim 8, further comprising:
at least one AI-based patient forecast provided by the AI software based on the time-series data, wherein the at least one AI-based patient forecast consists of at least one of at least one future health event of the patient, at least one medical or health diagnosis of the patient, at least one medication order or prescription order for the patient, and at least one future medical treatment, further observation, or future examinations of the patient.

10. The avatar assisted telemedicine platform system of claim 9, further comprising:
AI-based hardware executing and/or implementing the AI software to provide the at least one AI-based patient forecast, wherein the AI-based hardware consists of at least one selected from an AI-optimized CPU, an AI-optimized GPU, an AI-optimized IGP, an AI-specific accelerator, an AI-specific integrated circuit, and at least one combination thereof.

11. A avatar assisted telemedicine method for assisting a patient during a telemedicine service comprising a patient examination, the method comprising:
rendering, via the patient digital avatar according to claim 1, at least one communication in the native spoken-language of the patient such that the at least one communication assists the patient during at least one portion of the patient examination.

12. A avatar assisted telemedicine method for providing patient assistance during a telemedicine service comprising a patient examination, the method comprising:
receiving input data indicative of a native language of a patient from a patient digital device;
rendering or displaying at least one communication of a digital avatar via a GUI of the patient digital device, wherein the at least one communication is provided in the native language of the patient; and
assisting a queuing of the patient examination over the patient digital device via the at least one communication rendered or displayed by the digital avatar.

13. The avatar assisted telemedicine method of claim 12, further comprising:
completing a first job of the patient examination based on the assisted queuing of the patient examination over the digital device, wherein the assisted queuing is a sequence indicative of the patient examination comprising the first job of the patient examination and at least one second job of the patient examination.

14. The avatar assisted telemedicine method of claim 13, wherein the at least one communication consists of at least one instruction in the native spoken-language of the patient that is indicative of the first job of the patient examination.

15. The avatar assisted telemedicine method of claim 12, further comprising:
streaming the input data from the patient digital device, wherein the input data consists of audio data indicative of the native language of the patient.

16. The avatar assisted telemedicine method of claim 15, further comprising:
determining, identifying, and/or selecting the native language of the patient based on the streamed input data via artificial intelligence (AI) software.

17. The avatar assisted telemedicine method of claim 16, further comprising:
executing and/or implementing the AI software via an AI-based hardware to determine, identify, and/or select the native language of the patient, wherein the AI-based hardware consists of at least one selected from an AI-optimized CPU, an AI-optimized GPU, an AI-optimized IGP, an AI-specific accelerator, an AI-specific integrated circuit, and at least one combination thereof.

18. The avatar assisted telemedicine method of claim 12, further comprising:
streaming healthcare data of the patient collected during at least one portion of the patient examination from at least one sensor or monitoring device in communication with the patient digital device; and
processing and/or analyzing the streamed healthcare data of the patient collected during the at least one portion of the patient examination via artificial intelligence (AI) software.

19. The avatar assisted telemedicine platform method of claim 18, further comprising:
producing time-series data by indexing, listing, and/or graphing a series of data points in time order via the AI software, wherein the series of data points are based on the streamed healthcare data.

20. The avatar assisted telemedicine platform method of claim 20, further comprising:
predicting at least one AI-based patient forecast based on the time-series data via the AI software, wherein the at least one AI-based patient forecast consists of at least one of at least one future health event of the patient, at least one medical or health diagnosis of the patient, at least one medication order or prescription order for the patient, and at least one future medical treatment, further observation, or future examinations of the patient.
